(19) United States
(12) Patent Application Publication
(10) Pub. No.: US 2015/0307923 A1
(43) Pub. Date: Oct. 29, 2015

(54) MEGASPHAERA CEREVISIAE SYSTEM PROCESS CONTROL (SPC) PRIMERS, PROBES, AND METHODS

(71) Applicant: FRED HUTCHINSON CANCER RESEARCH CENTER, North, Seattle, WA (US)

(72) Inventors: David FREDRICKS, Seattle, WA (US); Daisy KO, Seattle, WA (US)

(21) Appl. No.: 14/651,067

(22) PCT Filed: Dec. 10, 2013
(86) PCT No.: PCT/US2013/074162

§ 371 Date: Jun. 10, 2015

Related U.S. Application Data
(60) Provisional application No. 61/735,415, filed on Dec. 10, 2012.

(51) Int. Cl. C12Q 1/68

(52) U.S. Cl. CPC C12Q 1/689; C12Q 2600/166

(57) ABSTRACT

Provided are system process control (SPC) compositions and methods for confirming DNA extraction and PCR amplification of a patient sample through the detection of an introduced control organism, such as Megasphaera cerevisiae, which is not otherwise present in a patient sample. Compositions of the present disclosure include primers, primer sets, and probes for specifically detecting a control organism introduced into a patient sample.

This application is being filed on 10 Dec. 2013, as a PCT International patent application, and claims priority to U.S. Provisional Patent Application No. 61/735,415, filed Dec. 10, 2012, the disclosure of which is incorporated by reference in its entirety.

### GOVERNMENT SPONSORED RESEARCH OR DEVELOPMENT

This disclosure was made in part in the course of research sponsored by the National Institute of Health, grant number U01 AI070801. The U.S. government has certain rights in this disclosure.

### SEQUENCE LISTING

The present application includes a Sequence Listing in electronic format as a txt file in ASCII format titled “54428_{—}0008 WOU1_SEQ_LIST_ST25.txt,” which was created on Dec. 10, 2013 and which has a size of 8,192 bytes. The contents of txt file 54428_{—}0008 WOU1_SEQ_LIST_ST25.txt” are incorporated by reference herein.

### BACKGROUND OF THE DISCLOSURE

1. Technical Field

The present disclosure is directed, generally, to the detection of bacteria associated with bacterial vaginosis in a patient sample. More specifically, disclosed herein are system process control (SPC) compositions and methods for confirming DNA extraction and PCR amplification of a patient sample through the detection of an introduced control organism, such as Megasphaera cerevisiae, which is not otherwise present in a patient sample. Compositions of the present disclosure include primers, primer sets, and probes for specifically detecting a control organism introduced into a patient sample.

2. Description of the Related Art

Bacterial vaginosis (BV) is a common condition, affecting millions of women annually (Wang, Ob. Gyns. 7:181-185 (2000)), and is associated with numerous health problems including pre-term labor and low birth weight (Leitich et al., Am. J. Obstet. Gynecol. 189:139-47 (2003) and Hillier et al., Clin. Infect. Dis. 20Sup2:S276-8 (1995)), pelvic inflammatory disease (Peipert et al., Am. J. Obstet. Gynecol. 184:856-63 (2001) and Hillier et al., Am. J. Obstet. Gynecol. 175:435-41 (1996)), and acquisition of human immunodeficiency virus (Martin et al., J. Infect. Dis. 180:1863-8 (1999) and Moodley et al., J. Infect. Dis. 185:69-73 (2002)). Malodorous vaginal discharge may be the only symptom of BV, and many affected individuals are asymptomatic (Klebanoff et al., Obstet. Gynecol. 104:267-72 (2004)).

Studies using cultivation methods have demonstrated that women with BV experience loss of vaginal lactobacilli and concomitant overgrowth of anaerobic and facultative bacteria. Several bacteria have been implicated in BV, such as Gardnerella vaginalis (Gardner and Dukes, Am. J. Obstet. Gynecol. 69:962-76 (1955)) and Mobiluncus curtisii (Spiegel, J. Infect. Dis. 148:817-22 (1983)), but these species are also found in subjects without BV, and thus are not specific markers for disease (Spiegel, Clin. Microbiol. Rev. 4:485-502 (1991)). For this reason, bacterial cultivation of vaginal fluid has not proven useful for the diagnosis of BV. Rather, clinical criteria or Gram stain analysis of vaginal fluid are employed for diagnosis. At least 3 of 4 elements must be present to fulfill Amsel clinical criteria for BV (Amsel et al., Am. J. Med. 74:14-22 (1983)), including presence of (1) thin, homogeneous, milky, vaginal discharge; (2) vaginal fluid pH greater than 4.5; (3) positive whiff test—production of fishy odor when 10% potassium hydroxide is added to a slide containing vaginal fluid; and (4) presence of clue cells (>20% of epithelial cells with adherent bacteria) on microscopic examination of vaginal fluid (Amsel et al., Am. J. Med. 74:14-22 (1983)). An alternative diagnostic approach employs Gram stain of vaginal fluid (Nugent score; Nugent et al., J. Clin. Microbiol. 29:297-301 (1991)) to distinguish normal vaginal flora (Gram-positive rods, lactobacilli) from BV flora (Gram-negative morphotypes; Spiegel et al., J. Clin. Microbiol. 18:170-7 (1983)).

Koch's postulates for establishing disease causation have not been fulfilled for any bacterium or group of bacteria associated with BV. BV responds to treatment with antibiotics such as metronidazole or clindamycin, but metronidazole has poor in vitro activity against G. vaginalis and M. curtisii. Relapse and persistence are common (Spiegel, Clin. Microbiol. Rev. 4:485-502 (1991)). Thus, the etiology and pathogenesis of BV remain poorly understood, and management can be challenging.

Only a fraction of the bacteria present in most microbial ecosystems are amenable to propagation in the laboratory (Hugenholtz et al., J. Bacteriol. 180:4765-74 (1998)). Bacteria in complex microbial communities can be identified by characterizing their ribosomal RNA genes (rDNA), an approach that has the advantage of detecting fastidious or cultivation-resistant organisms (Pace, Science 276:734-40 (1997)).

Fredricks and Fiedler recently described a methodology for the identification of bacteria present in vaginal fluid samples using an approach that employs molecular methods, in particular PCR amplification of patient samples (U.S. Pat. No. 7,625,704, which is incorporated herein by reference in its entirety). PCR assays for the detection of bacterial pathogens are an appealing approach due to their potential for rapid, sensitive, and accurate diagnosis of bacterial infections. A potential shortcoming of these techniques is the possibility of false negatives owing to (a) the incomplete lysis, prior to PCR amplification, of bacteria present in the patient sample and/or (b) the presence of one or more PCR inhibitors within the patient sample that prevent the amplification of a bacterium's genetic material.

What is critically needed in the art are technologies for assessing the PCR reaction to ensure that bacteria present in a patient sample are reliably detected thereby reducing the possibility of misdiagnosing a bacterial infection in a patient.

### SUMMARY OF THE DISCLOSURE

The present disclosure achieves these and other related needs by providing system process control (SPC) compositions and methods for confirming the efficiency of DNA extraction and PCR amplification of a patient sample. SPC compositions and methods disclosed herein can employ at least one forward and reverse primer pair and at least one control organism, which is not otherwise present in a patient sample. The SPC compositions and methods can also include a probe for detecting an amplified region of the genome of the at least one control organism. Exemplified herein are compositions and methods that employ at least one primer pair, and optionally at least one probe, for specifically amplifying and detecting DNA from a Megasphaera cerevisiae control bacterium, which is introduced into a patient sample prior to cell lysis and PCR amplification.

In certain embodiments, the SPC methods include: (a) introducing a control bacterium into a patient sample, (b) carrying out a PCR reaction on the patient sample to generate a PCR amplicon that comprises a region of the control bacterium's genome, wherein the PCR reaction uses at least one primer pair including a forward primer and a reverse primer wherein each of the forward and reverse primers is complementary to a region of the control bacterium's genome, and (c) detecting the PCR amplicon. By the presently disclosed SPC methods, the control bacterium can be a Megasphaera cerevisiae species.

The region of the control bacterium's genome that is amplified by the SPC methods can be at least a portion of the control bacterium's ribosomal RNA (rRNA). Within certain aspects of these methods, the amplified region of the control bacterium's rRNA gene can include at least a portion of an internal transcribed spacer (ITS) region. The amplified region of the control bacterium's rRNA gene region can, optionally, also include at least a portion of a 16S rRNA gene and/or at least a portion of a 23S rRNA gene. Typically, the PCR amplicon amplified by the present SPC methods is between about 50 bp and about 1000 bp, or between about 60 bp and about 600 bp, or between about 70 bp and about 400 bp, or between about 80 bp and about 300 bp.

The PCR amplicon can be detected by hybridization of the amplicon to a probe, such as a radiolabeled or a fluorescently labeled probe. In compositions and methods in which the control bacterium is a Megasphaera cerevisiae species and the region of the control bacterium's genome that is amplified by the PCR reaction is at least a portion of the control bacterium's ribosomal RNA (rRNA), the probe can include the nucleotide sequence presented in SEQ ID NO: 7 (i.e., 5′-ATAGTATATGTTGAAAGACATGTAGTATGAGCGCAG-3′). Exemplified herein is a probe comprising the sequence of SEQ ID NO: 7 further comprising the fluorophore Atto647N coupled to the probe's 5′ end and the fluorophore BHQ2 coupled to the probe's 3′ end. The present SPC methods can, optionally, include sequencing at least a portion of the PCR amplicon that is generated by the PCR reaction.

The forward and reverse primers used in the presently disclosed methods can both be complementary to a control bacterium's ITS rRNA gene. In other aspects, the forward primer can be complementary to a control bacterium's ITS rRNA gene and the reverse primer can be complementary to a control bacterium's 23S rRNA gene. In other aspects, the forward primer can be complementary to a control bacterium's 16S rRNA gene and the reverse primer can be complementary to a control bacterium's ITS rRNA gene. In other aspects, the forward primer can be complementary to a control bacterium's 16S rRNA gene and the reverse primer can be complementary to a control bacterium's 23S rRNA gene.

In compositions and methods in which the control bacterium is a Megasphaera cerevisiae species and the region of the control bacterium's genome that is amplified by the PCR reaction is at least a portion of the control bacterium's ribosomal RNA (rRNA), the forward primer and the reverse primer can be complementary to at least a portion of the Megasphaera cerevisiae rRNA ITS region presented herein as SEQ ID NO: 8. Within these aspects of the presently disclosed compositions and methods, the portion of the Megasphaera cerevisiae rRNA ITS region that is amplified can be between about 50 bp and about 1000 bp, or between about 60 bp and about 600 bp, or between about 70 bp and about 400 bp, or between about 80 bp and about 300 bp of the sequence presented herein as SEQ ID NO: 8.

For example, the forward primer can comprise at least a portion of the nucleotide sequence 5′-CGAGTCACTTATGCCGGATAT-3′ (SEQ ID NO: 1) and/or the reverse primer can comprise at least a portion of a nucleotide sequence selected from 5′-CCTTACTGTATCTCTACTTCGC-3′ (SEQ ID NO: 2), 5′-CCTAAGTGATTGGGTTGAGTC-3′ (SEQ ID NO: 3), 5′-TTGGTTGTTCCAGAATGCCGA-3′ (SEQ ID NO: 4), 5′-TGATTCATTCCAGATGAGAGAAG-3′ (SEQ ID NO: 5), and 5′-TGTCTTCACCTTGTATATATTAGAG-3′ (SEQ ID NO: 6). Other forward and reverse primer sequences that can be used to amplify at least a portion of a Megasphaera cerevisiae rRNA ITS region are also contemplated by the present disclosure.

Within certain embodiments disclosed herein, the compositions and methods employ primer sets that include a forward and reverse primer pair wherein the primer sets can be selected from: (SEQ ID NO: 1 and SEQ ID NO: 2), (SEQ ID NO: 1 and SEQ ID NO: 3), (SEQ ID NO: 1 and SEQ ID NO: 4), (SEQ ID NO: 1 and SEQ ID NO: 5), and (SEQ ID NO: 1 and SEQ ID NO: 6). Within related aspects of these embodiments, each primer of a primer pair can contain at least a portion of each of the recited sequences. For example, each primer of a primer pair can contain at least 12, 13, 14, 15, 16, 17, 18, 19, 20, or 21 contiguous nucleotides of each of the recited sequences. Alternatively or additionally, each primer of a primer pair can be at least about 80% identical with, at least about 85% identical with, at least about 90% identical with, at least about 95% identical with, and/or at least about 98% identical with each of the recited sequences.

Primers disclosed herein were designed to be used in PCR-based methods for detecting a control bacterium's DNA in a patient sample. Thus, these primers specifically bind to a control bacterium's DNA but not to DNA, including non-control bacterial DNA, which is present in a patient sample. Thus, each primer of the primer pair specifically binds only to a control bacterium's DNA in the presence of a non-control bacterium's DNA and/or a patient's DNA, which is present in a patient sample. As demonstrated herein, primers of the present disclosure permit the amplification of a control bacterium's DNA in a patient sample wherein the non-control bacterium's DNA and/or the patient's DNA is present in greater than 100,000-fold, 500,000-fold, 2,500,000-fold, or 10,000,000-fold mass excess over the amount of control bacterium's DNA.

Also provided are methods for identifying a primer set capable of detecting a control bacterium in a patient sample. These methods comprise: (a) obtaining the nucleic acid sequence of at least a portion of said control bacterium's rRNA gene, (b) designing a forward primer capable of hybridizing with said nucleic acid sequence at a specific site in a 16S region or an ITS region of said said rRNA gene, (c) designing a reverse primer capable of hybridizing with said nucleic acid sequence at a specific site in an ITS region or a 23S region of said rRNA gene, wherein said reverse primer hybridizes to a specific site that is 3′ to the specific site to which the forward primer hybridizes, and (d) determining whether said forward primer and said reverse primer are capable of generating a PCR amplicon that is useful for identifying said control bacterium's DNA in a PCR reaction containing said control bacterium. The control bacterium can be Megasphaera cerevisiae and at least one of the forward primer and the reverse primer are capable of hybridizing to the Megasphaera cerevisiae ITS sequence presented in SEQ ID NO: 8.

These and other embodiments, features and advantages of the disclosure will become apparent from the following figures, detailed description, and the appended claims set forth herein below.

### BRIEF DESCRIPTION OF THE DRAWINGS

FIG. 1 is a bar graph showing threshold cycle (Ct) values as a function of M cerevisiae genomic DNA concentration (1000 pg, 100 pg, 10 pg, 1 pg, 0.5 pg, and 0.1 pg) for quantitative PCR (qPCR) reactions using five primer combinations in which the same forward primer (SEQ ID NO: 1) was used in combination with one of five reverse primers (SEQ ID NOs: 2-6) to generate M. cerevisiae DNA amplicons. These data demonstrate that each primer combination was able to amplify down to at least 0.1 pg of M. cerevisiae DNA.

FIG. 2 is a bar graph showing an increase in average melting temperature (Tm) as a function of increasing amplicon size for quantitative PCR (qPCR) reactions using five primer combinations in which the same forward primer (SEQ ID NO: 1) was used in combination with one of five reverse primers (SEQ ID NOs: 2-6) to generate M. cerevisiae DNA amplicons of progressively increasing size.

FIG. 3 is a graph showing normalized Rn (i.e., delta Rn) as a function of cycle number for amplicons derived from multiplex qPCR reactions with the reverse primer of SEQ ID NO: 4 (ITS521) and M. cerevisiae probe (SEQ ID NO: 7) in multiplex qPCR reactions targeting 0.5 pg of M. cerevisiae genomic DNA (SPC), 10⁶ 16 S plasmid copies of Megasphaera types 1 and 2 (high mega), and 10⁶ 16 S copies of the Clostridium-like bacterial vaginosis bacterium BVAB2 (high by). Delta Rn was obtained by normalizing the reporter signal (Rn) to a fluorescence signal from the ROX™ reference fluorescent dye and subtracting the baseline from Rn (i.e., ΔRn=Rn−baseline)).

FIG. 4 is a graph showing normalized Rn (i.e., delta Rn) as a function of cycle number for amplicons derived from multiplex qPCR reactions with the reverse primer of SEQ ID NO: 4 (ITS521) and M. cerevisiae probe (SEQ ID NO: 7) in multiplex qPCR reactions targeting 0.5 pg of M. cerevisiae genomic DNA (SPC), 100 16S plasmid copies of Megasphaera types 1 and 2 (low mega), and 100 16S copies of the Clostridium-like bacterial vaginosis bacterium BVAB2 (low by). Delta Rn was obtained by normalizing the reporter signal (Rn) to a fluorescence signal from the ROX™ reference fluorescent dye and subtracting the baseline from Rn (i.e., ΔRn=Rn−baseline)).

FIG. 5 is a graph showing normalized Rn (i.e., delta Rn) as a function of cycle number for amplicons derived from multiplex qPCR reactions with the reverse primer of SEQ ID NO: 5 (ITS576) and M. cerevisiae probe (SEQ ID NO: 7) in multiplex qPCR reactions targeting 0.5 pg of M. cerevisiae genomic DNA (SPC), 10⁶ 16 S plasmid copies of Megasphaera types 1 and 2 (high mega), and 10⁶ 16 S copies of the Clostridium-like bacterial vaginosis bacterium BVAB2 (high by). Delta Rn was obtained by normalizing the reporter signal (Rn) to a fluorescence signal from the ROX™ reference fluorescent dye and subtracting the baseline from Rn (i.e., ΔRn=Rn−baseline)).

FIG. 6 is a graph showing normalized Rn (i.e., delta Rn) as a function of cycle number for amplicons derived from multiplex qPCR reactions with the reverse primer of SEQ ID NO: 5 (ITS576) and M. cerevisiae probe (SEQ ID NO: 7) in multiplex qPCR reactions targeting 0.5 pg of M. cerevisiae genomic DNA (SPC), 100 16S plasmid copies of Megasphaera types 1 and 2 (low mega), and 100 16S copies of the Clostridium-like bacterial vaginosis bacterium BVAB2 (low by). Delta Rn was obtained by normalizing the reporter signal (Rn) to a fluorescence signal from the ROX™ reference fluorescent dye and subtracting the baseline from Rn (i.e., ΔRn=Rn−baseline)).

### DETAILED DESCRIPTION OF THE DISCLOSURE

As discussed above, the present disclosure provides system process control (SPC) compositions and methods for confirming the efficiency of cell lysis, DNA extraction, and PCR amplification of a patient sample. False negative PCR results can occur if cells are not lysed, if DNA and/or RNA is not successfully extracted, and/or if one or more PCR inhibitor is present. Thus, the presently disclosed compositions and methods permit a determination of whether there is a false negative PCR reaction. A control bacterium is added to a patient sample to be processed for nucleic acid extraction and PCR. The cells are lysed and nucleic acids are extracted and submitted to a PCR reaction. PCR is performed using primers and probes that target a region of the control bacterium's genome, such as its rRNA gene. If the cell lysis, nucleic acid extraction, and PCR amplification are successful, then the control bacterium will be detected by observing the resulting PCR amplicon, thereby ruling out a false negative PCR reaction for the second target of interest. If the control bacterium is not detected, this suggests a problem is present with either the DNA extraction step or the PCR step—a result that warrants re-testing of the patient sample.

The bacterium Megasphaera cerevisiae is a cause of beer spoilage in breweries and is not part of the human microbiota, yet it is related to bacteria found in the human body. Megasphaera cerevisiae can, therefore, be as a used surrogate for the ability to detect human microbes and is a suitable control bacterium in the compositions and methods presented herein.

The present disclosure will be best understood by reference to the following definitions:

### DEFINITIONS

As used herein, the term “isolated” means that the referenced material is removed from its native environment, e.g., a cell or fungus. Thus, an isolated biological material can be free of some or all cellular components, i.e., components of the cells in which the native material occurs naturally (e.g., cytoplasmic or membrane component). A material shall be deemed isolated if it is present in a cell extract or supernatant. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined or proximal to non-coding regions (but may be joined to its native regulatory regions or portions thereof), or to other genes, located upstream or downstream of the gene contained by the isolated nucleic acid molecule when found in the chromosome. In yet another embodiment, the isolated nucleic acid lacks one or more introns. Isolated nucleic acid molecules include sequences inserted into plasmids, cosmids, artificial chromosomes, and the like, i.e., when it forms part of a chimeric recombinant nucleic acid construct. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e. contaminants, including native materials from which the material is obtained. For example, a purified fungal DNA is preferably substantially free of cell or culture components, including tissue culture components, contaminants, and the like. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

As used herein, the terms “include” and “comprise” are used synonymously. It should be understood that the terms “a” and “an” as used herein refer to “one or more” of the enumerated components. The use of the alternative (e.g., “or”) should be understood to mean either one, both, or any combination thereof of the alternatives.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean±20% of the indicated range, value, or structure, unless otherwise indicated.

In a specific embodiment, the term “about” or “approximately” means within a statistically meaningful range of a value. Such a range can be within an order of magnitude, preferably within 50%, more preferably within 20%, more preferably still within 10%, and even more preferably within 5% of a given value or range. The allowable variation encompassed by the term “about” or “approximately” depends on the particular system under study, and can be readily appreciated by one of ordinary skill in the art.

The term “contig” as used herein, refers to one of a set of overlapping clones that represent a continuous region of DNA. However, in certain embodiments, “contig” also refers to a contiguous sequence constructed from many clone sequences or PCR products, and herein, is used synonymously with the term “sequence.”

Techniques to isolate and modify specific nucleic acids and proteins are well known to those of skill in the art. In accordance with the present disclosure there may be employed conventional molecular biology, microbiology, and recombinant DNA techniques within the skill of the art. Such techniques are explained fully in the literature. See, e.g., Sambrook et al., Molecular Cloning: A Laboratory Manual, Second Edition (Cold Spring Harbor, N.Y.: Cold Spring Harbor Laboratory Press, 1989) (“Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. 1985); Transcription And Translation (B. D. Hames & S. J. Higgins, eds. 1984); Animal Cell Culture (R. I. Freshney, ed. 1986); Immobilized Cells And Enzymes (IRL Press, 1986); Perbal, “A Practical Guide To Molecular Cloning” (Ausubel, F. M. et al. eds., 1984); Current Protocols in Molecular Biology (John Wiley & Sons, Inc., 1994). These techniques include site directed mutagenesis employing oligonucleotides with altered nucleotides for generating PCR products with mutations (e.g., the “Quikchange” kit manufactured by Stratagene).

The terms “nucleic acid” and “oligonucleotide,” as used herein, refer to polydeoxyribonucleotides (containing 2-deoxy-D-ribose), to polyribonucleotides (containing D-ribose), and to any other type of polynucleotide which is an N glycoside of a purine or pyrimidine base. There is no intended distinction in length between the terms “nucleic acid” and “oligonucleotide”, and these terms will be used interchangeably. These terms refer only to the primary structure of the molecule. Thus, these terms include double- and single-stranded DNA, as well as double- and single-stranded RNA. For use in the present disclosure, an oligonucleotide also can comprise non-purine or non-pyrimidine nucleotide analogs. The length of a nucleic acid sequence is referred to as the number of “base pairs (bp)” present in the double-stranded nucleic acid sequence.

The nucleic acid molecules of sequences disclosed herein are written according to The International Union of Pure and Applied Chemistry (IUPAC) DNA codes. Specifically, “A” is Adenine, “C” is Cytosine, “G” is Guanine, “T” is Thymine, “U” is Uracil, “R” is any Purine (A or G), “Y” is any Pyrimidine (C, T, or U), “M” is C or A, “K” is T, U, or G, “W” is T, U, or A, “S” is C or G, “B” is C, T, U, or G (not A), “D” is A, T, U, or G (not C), “H” is A, T, U, or C (not G), “V” is A, C, or G (not T, not U), and “N” is any base (A, C, G, T, or U).

In certain embodiments, the amount of control bacterial DNA introduced into a patient sample is described in terms of the “fold-excess” of human DNA and/or non-control bacterial DNA over the amount of control bacterial DNA present in the same sample. For example, if 1 μg of human genomic DNA is present in a sample that has 0.001 μg of control bacterial DNA, then the human DNA is understood to be in 1000-fold excess of the control bacterial DNA.

The term “primer,” as used herein, refers to an oligonucleotide capable of acting as a point of initiation of DNA synthesis under conditions in which synthesis of a primer extension product complementary to a nucleic acid strand is induced, i.e., either in the presence of four different nucleoside triphosphates and an agent for extension (e.g., a DNA polymerase or reverse transcriptase) in an appropriate buffer and at a suitable temperature. A primer is preferably a single-stranded DNA. The appropriate length of a primer depends on the intended use of the primer but typically ranges from 6 to 50 nucleotides, preferably from 15-35 nucleotides. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein. As used herein, a “forward primer” is understood to mean a primer that is capable of hybridizing to a region of DNA along the 5′ (coding) strand of DNA. A “reverse” primer is understood to mean a primer that is capable of hybridizing to a region of DNA along the 3′ (non-coding) strand of DNA.

Primers can incorporate additional features which allow for the detection or immobilization of the primer but do not alter the basic property of the primer, that of acting as a point of initiation of DNA synthesis. For example, primers may contain an additional nucleic acid sequence at the 5′ end which does not hybridize to the target nucleic acid, but which facilitates cloning of the amplified product. The region of the primer which is sufficiently complementary to the template to hybridize is referred to herein as the hybridizing region.

In certain embodiments, the term “primer” is also intended to encompass the oligonucleotides used in ligation-mediated amplification processes, in which one oligonucleotide is “extended” by ligation to a second oligonucleotide which hybridizes at an adjacent position. Thus, the term “primer extension”, as used herein, refers to both the polymerization of individual nucleoside triphosphates using the primer as a point of initiation of DNA synthesis and to the ligation of two oligonucleotides to form an extended product.

The term “probe” or “primer” includes naturally occurring or recombinant or chemically synthesized single- and/or double-stranded nucleic acids. They can be labeled for detection by nick translation, Klenow fill-in reaction, PCR or other methods well known in the art. A probe or primer can be an oligonucleotide and can comprise any number of nucleotides and in some embodiments can comprise, for example, at least 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 60, 70, 80. 90, 100, 125, 150, 175, 200, 250, 300 nucleotides or more as appropriate for the particular assay in which it will be used. Probes and primers of the present disclosure, their preparation and/or labeling are described in Sambrook et al., 1989 and Ausubel et al., Current Protocols in Molecular Biology, John Wiley & Sons, New York, N.Y., 1989) both of which are incorporated herein by reference in their entirety for these teachings.

In particular embodiments, the probes and/or primers of this disclosure can have at least about 50%, 60%, 70%, 75%, 80%, 85%, 90%, 95%, 98% or more nucleic acid sequence homology with the sequences specifically disclosed herein. The term “homology” as used herein refers to a degree of similarity between two or more sequences. There may be partial homology or complete homology (i.e., identity). A partially complementary sequence that at least partially inhibits an identical sequence from hybridizing to a target nucleic acid is referred to as “substantially homologous.” The inhibition of hybridization of the completely complementary sequence to the target sequence can be examined using a hybridization assay (e.g., Southern or Northern blot, solution hybridization and the like) under conditions of low stringency.

A “primer set” or “primer pair” refers to a specific combination of a forward primer and one or more reverse primers. Some “primer sets” or “primer pairs” may include, for example, one forward primer and one reverse primers (e.g., a primer set comprising SEQ ID NO: 1 and at least one of SEQ ID NO: 2, SEQ ID NO: 3, SEQ ID NO: 4, SEQ ID NO: 5, and/or SEQ ID NO: 6). “Primer pairs” that are suitable for the detection of a Megasphaera cerevisiae species that is introduced into a patient sample include forward and reverse primer pairs wherein the primer sets may be selected from (SEQ ID NO: 1 and SEQ ID NO: 2), (SEQ ID NO: 1 and SEQ ID NO: 3), (SEQ ID NO: 1 and SEQ ID NO: 4), (SEQ ID NO: 1 and SEQ ID NO: 5), and (SEQ ID NO: 1 and SEQ ID NO: 6). The “primer set” or “primer pair” may be used in a PCR reaction to generate a specific PCR product or amplicon.

Oligonucleotides can be prepared by any suitable method, including direct chemical synthesis by a method such as the phosphotriester method of Narang et al. (1979) Meth. Enzymol. 68:90-99; the phosphodiester method of Brown et al., (1979) Meth. Enzymol. 68:109-151; the diethylphosphoramidite method of Beaucage et al. (1981) Tetrahedron Lett. 22:1859-1862; and the solid support method of U.S. Pat. No. 4,458,066, each incorporated herein by reference. A review of synthesis methods of conjugates of oligonucleotides and modified nucleotides is provided in Goodchild (1990) Bioconjugate Chemistry 1(3):165-187, incorporated herein by reference.

In the methods of the present disclosure that recite the use of a primer pair to amplify a target nucleic acid, it is understood that such a method is exemplary of one of a variety of methods for amplification of nucleic acid, some of which employ primers and primer pairs and some of which amplify by other means, as is well known in the art. Thus, the methods of this disclosure wherein amplification of nucleic acid is described are not intended to be limited to amplification methods employing only primer pairs and other such amplification methods are described herein and are well known in the art. The terms “target, “target sequence”, “target region”, and “target nucleic acid,” as used herein, are synonymous and refer to a region or subsequence of a nucleic acid which is to be amplified or detected.

Furthermore, the terms “under conditions whereby nucleic acid amplification can occur” and “under conditions whereby nucleic acid hybridization can occur” and variations thereof would be well recognized by one of ordinary skill in the art to mean conditions employing specific reagents, solutions, temperature, pH and/or physical conditions that allow for amplification of nucleic acid and/or hybridization of nucleic acid according to protocols well known in the art.

Claims that refer to conditions whereby the amount of amplified nucleic acid or hybridized nucleic acid can be quantitated describe conditions that are also well known to the ordinary person of skill in the art. In particular, methods of determining the amount of amplified nucleic acid are well known for such protocols as PCR (e.g., quantitative PCR or qPCR) and other amplification protocols and method of determining the amount of hybridized nucleic acid both semi-quantitatively and quantitatively are also well known in the art and as described herein.

The term “amplicon” as used herein, refers to the DNA sequence generated by a PCR or qPCR reaction. “Amplicon” may further be used synonymously with the term “PCR product.” The term “amplification reaction” refers to any chemical reaction, including an enzymatic reaction, which results in increased copies of a template nucleic acid sequence or results in transcription of a template nucleic acid. Amplification reactions include reverse transcription and the polymerase chain reaction (PCR), including Real Time PCR (see U.S. Pat. Nos. 4,683,195 and 4,683,202; PCR Protocols: A Guide to Methods and Applications (Innis et al., eds, 1990)). Exemplary “amplification reactions conditions” or “amplification conditions” typically comprise either two or three step cycles. Two step cycles have a denaturation step followed by a hybridization/elongation step. Three step cycles comprise a denaturation step followed by a hybridization step followed by a separate elongation step.

Polymerase chain reaction (PCR) is a method that allows exponential amplification of short DNA sequences (usually 100 to 600 bases) within a longer double stranded DNA molecule. PCR entails the use of a pair of primers, each about 20 nucleotides in length, which are complementary to a defined sequence on each of the two strands of the DNA. These primers are extended by a DNA polymerase so that a copy is made of the designated sequence. After making this copy, the same primers can be used again, not only to make another copy of the input DNA strand but also of the short copy made in the first round of synthesis. This leads to logarithmic amplification. Since it is necessary to raise the temperature to separate the two strands of the double strand DNA in each round of the amplification process, a major step forward was the discovery of a thermo-stable DNA polymerase (Taq polymerase) that was isolated from Thermus aquaticus, a bacterium that grows in hot pools; as a result it is not necessary to add new polymerase in every round of amplification. After several (often about 40) rounds of amplification, the PCR product is analyzed on an agarose gel and is abundant enough to be detected with an ethidium bromide stain.

As used herein, a “polymerase” refers to an enzyme that catalyzes the polymerization of nucleotides. Generally, the enzyme will initiate synthesis at the 3′-end of the primer annealed to a nucleic acid template sequence. “DNA polymerase” catalyzes the polymerization of deoxyribonucleotides. Known DNA polymerases include, for example, Pyrococcus furiosus (Pfu) DNA polymerase (Lundberg et al., (1991) Gene 108:1), E. coli DNA polymerase I (Lecomte and Doubleday (1983) Nucleic Acids Res. 11:7505), T7 DNA polymerase (Nordstrom et al. (1981) J. Biol. Chem. 256:3112), Thermus thermophilus (Tth) DNA polymerase (Myers and Gelfand (1991) Biochemistry 30:7661), Bacillus stearothermophilus DNA polymerase (Stenesh and McGowan (1977) Biochim Biophys Acta 475:32), Thermococcus litoralis (Tli) DNA polymerase (also referred to as Vent DNA polymerase, Cariello et al. (1991) Nucleic Acids Res 19:4193), Thermotoga maritima (Tma) DNA polymerase (Diaz and Sabino (1998) Braz J. Med. Res 31:1239), Thermus aquaticus (Taq) DNA polymerase (Chien et al., (1976) J. Bacteoriol 127:1550), Pyrococcus kodakaraensis KOD DNA polymerase (Takagi et al. (1997) Appl. Environ. Microbiol. 63:4504), JDF-3 DNA polymerase (Patent application WO 0132887), and Pyrococcus GB-D (PGB-D) DNA polymerase (Juncosa-Ginesta et al. (1994) Biotechniques 16:820). The polymerase activity of any of the above enzymes can be determined by means well known in the art.

In other embodiments, real-time PCR, also called quantitative real time PCR, quantitative PCR (Q-PCR/qPCR), or kinetic polymerase chain reaction, is a laboratory technique based on PCR, which is used to amplify and simultaneously quantify a targeted DNA molecule. qPCR enables both detection and quantification (as absolute number of copies or relative amount when normalized to DNA input or additional normalizing genes) of a specific sequence in a DNA sample. For example, in the embodiments disclosed herein, qPCR may be used to quantify the amount of fungal DNA in a patient sample. The procedure follows the general principle of PCR; its key feature is that the amplified DNA is quantified as it accumulates in the reaction in real time after each amplification cycle. Two common methods of quantification are the use of fluorescent dyes that intercalate with double-stranded DNA, and modified DNA oligonucleotide probes that fluoresce upon binding to complementary DNA (such as with molecular beacons) or with completion of each PCR cycle (such as with dual labeled probes rendered more fluorescent with the 5′ exonuclease activity of polymerase enzymes).

“Endpoint PCR” is understood to mean a semi-quantitative approach to measuring relative amounts of template (DNA) in a sample involving the measurement of the amount of PCR product present at the end of a PCR reaction. In certain embodiments of the present disclosure, end-point PCR is performed by resolving the PCR amplicon on an agarose gel and staining the gel with an “intercalating” dye, such as, for example, ethidium bromide. Ethidium bromide binds between the bases of the DNA helix. When it is inserted into the DNA, it becomes much more fluorescent when exposed to ultraviolet light as compared to ethidium bromide just in solution. This characteristic of ethidium bromide permits semi-quantitative measurements of the amount of DNA in the PCR product by measuring the degree of fluorescence of the PCR product in the gel.

A reverse transcriptase PCR amplification procedure may be performed in order to quantify the amount of mRNA amplified. Methods of reverse transcribing RNA into cDNA are well known and described in Sambrook et al., 1989.

Alternative methods for reverse transcription utilize thermostable, RNA-dependent DNA polymerases. These methods are described, for example, in PCT Patent Publication No. WO 90/107641, incorporated herein by reference in its entirety. Polymerase chain reaction methodologies are well known in the art. Modifications to amplification assays such as PCR to allow for quantitative analysis of the amplified products are also well known in the art and such protocols and reagents are available in various commercial embodiments.

Another method for amplification is the ligase chain reaction (“LCR”), disclosed in Eur. Pat. Appl. No. 320308, incorporated herein by reference in its entirety. In LCR, two complementary probe pairs are prepared and in the presence of the target sequence, each pair will bind to opposite complementary strands of the target such that they abut. In the presence of a ligase, the two probe pairs will link to form a single unit. By temperature cycling, as in PCR, bound ligated units dissociate from the target and then serve as “target sequences” for ligation of excess probe pairs. U.S. Pat. No. 4,883,750 describes a method similar to LCR for binding probe pairs to a target sequence.

Q beta Replicase (QβR), described in PCT Patent Application No. PCT/US87/00880, incorporated herein by reference, can also be used as still another amplification method in the present disclosure. In this method, a replicative sequence of RNA that has a region complementary to that of a target is added to a sample in the presence of an RNA polymerase. The polymerase will copy the replicative sequence that can then be detected.

An isothennal amplification method, in which restriction endonucleases and ligases are used to achieve the amplification of target molecules that contain nucleotide 5′-[alphathio]triphosphates in one strand of a restriction site may also be useful in the amplification of nucleic acids in the present disclosure.

Strand Displacement Amplification (SDA), described in U.S. Pat. Nos. 5,455,166; 5,648,211; 5,712,124; and 5,744,311, each incorporated herein by reference, is another method of carrying out isothermal amplification of nucleic acids that which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present.

The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification method, as described in Great Britain Patent 2202328, and in PCT Patent Application No. PCT/US89/01025, each of which is incorporated herein by reference in its entirety, may be used in accordance with the present disclosure. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact, available to be bound by excess probe. Cleavage of the labeled probe signals the presence of the target sequence.

Other nucleic acid amplification procedures include transcription-based amplification systems (TAS), including nucleic acid sequence based amplification (NASBA) and 3SR (PCT Patent Publication No. WO 88/110315, incorporated herein by reference). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer that has target specific sequences. Following polymerization, DNA/RNA hybrids are digested with RNase H while double stranded DNA molecules are heat denatured again. In either case the single stranded DNA is made fully double-stranded by addition of second target specific primer, followed by polymerization. The double-stranded DNA molecules are then multiply transcribed by an RNA polymerase such as T7, T3 or SP6. In an isothermal cyclic reaction, the RNAs are reverse transcribed into single stranded DNA, which is then converted to double-stranded DNA, and then transcribed once again with an RNA polymerase such as T7, T3 or SP6. The resulting products, whether truncated or complete, indicate target specific sequences.

European Patent Application No. 329822 (incorporated herein by reference in its entirety) discloses a nucleic acid amplification process involving cyclically synthesizing single stranded RNA (ssRNA), ssDNA, and double-stranded DNA (dsDNA), which may be used in accordance with the present disclosure. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (dsDNA) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

PCT Patent Publication No. WO 89/06700 (incorporated herein by reference in its entirety) discloses a nucleic acid sequence amplification scheme based on the hybridization of a promoter/primer sequence to a target single-stranded DNA (ssDNA) followed by transcription of many RNA copies of the sequence. This scheme is not cyclic, i.e., new templates are not produced from the resultant RNA transcripts. Other amplification methods include “RACE” and “one-sided PCR” (Frohman, 1990, incorporated by reference).

Methods based on ligation of two (or more) oligonucleotides in the presence of nucleic acid having the sequence of the resulting “di-oligonucleotide,” thereby amplifying the di-oligonucleotide, may also be used in the amplification step of the present disclosure.

Following any amplification, it may be desirable to separate the amplification product from the template and the excess primer for the purpose of determining whether specific amplification has occurred. In one embodiment, amplification products are separated by agarose, agarose-acrylamide, or polyacrylamide gel electrophoresis using standard methods. See, e.g., Sambrook et al., 1989.

Alternatively, chromatographic techniques may be employed to effect separation. There are many kinds of chromatography that can be used in the present disclosure such as, for example, adsorption, partition, ion exchange and molecular sieve, as well as many specialized techniques for using them including column, paper, thin-layer and gas chromatography.

Amplification products must be visualized in order to confirm amplification of the target sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation. In one embodiment, visualization is achieved indirectly.

Following separation of amplification products, a labeled nucleic acid probe is brought into contact with the amplified target sequence. The probe preferably is conjugated to a chromophore but may be radio-labeled. In another embodiment, the probe is conjugated to a binding partner, such as an antibody or biotin, and the other member of the binding pair carries a detectable moiety.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which only fully complementary nucleic acid strands will hybridize are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989) and Wetmur, Critical Review in Biochem. and Mol. Biol. 26(3-4):227-259 (1991); both incorporated herein by reference).

A substantially homologous sequence or hybridization probe will compete for and inhibit the binding of a completely homologous sequence to the target sequence under conditions of low stringency, as this term is known in the art. This is not to say that conditions of low stringency are such that non-specific binding is permitted; low stringency conditions require that the binding of two sequences to one another be a specific (i.e., selective) interaction. The absence of non-specific binding can be tested by the use of a second target sequence that lacks even a partial degree of complementarity (e.g., less than about 30% identity). In the absence of nonspecific binding, the probe will not hybridize to the second non-complementary target sequence.

The term “stringent” as used herein refers to hybridization conditions that are commonly understood in the art to define the conditions of the hybridization procedure. Stringency conditions can be low, high or medium, as those terms are commonly known in the art and well recognized by one of ordinary skill. In various embodiments, stringent conditions can include, for example, highly stringent (i.e., high stringency) conditions (e.g., hybridization to filter-bound DNA in 0.5 M NaHP0₄, 7% sodium dodecyl sulfate (SDS), 1 mM EDTA at 65° C., and washing in 0.1×SSC/0.1% SDS at 68° C.), and/or moderately stringent (i.e., medium stringency) conditions (e.g., washing in 0.2×SSC/0.1% SDS at 42° C.).

Another example of stringency conditions can be hybridization in 25% formamide, 5×SSC, 5×Denhardt's solution, with 100 μg/ml of single stranded DNA and 5% dextran sulfate at 42° C., with wash conditions of 25% formamide, 5×SSC, 0.1% SDS at 42° C. for 15 minutes, to allow hybridization of sequences of about 60% homology.

More stringent conditions (e.g., high stringency) can be represented by a wash stringency of 0.3 M NaCl, 0.03 M sodium citrate, 0.1% SDS at 60° C. or even 70° C. using a standard in situ hybridization assay. See, e.g., Sambrook et al., 1989.

In other embodiments, detection is by Southern blotting and hybridization with a labeled probe. The techniques involved in Southern blotting are well known to those of skill in the art and can be found in many standard books on molecular protocols (Sambrook et al., 1989). Briefly, amplification products are separated by gel electrophoresis. The gel is then contacted with a membrane, such as nitrocellulose, permitting transfer of the nucleic acid and noncovalent binding. Subsequently, the membrane is incubated with a chromophore-conjugated probe that is capable of hybridizing with a target amplification product. Detection is by exposure of the membrane to x-ray film or ion-emitting detection devices. One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present disclosure.

As used herein, a primer is “specific,” for a target sequence if, when used in an amplification reaction under sufficiently stringent conditions, the primer hybridizes primarily only to the target nucleic acid. Typically, a primer is specific for a target sequence if the primer-target duplex stability is greater than the stability of a duplex formed between the primer and any other sequence found in the sample. One of skill in the art will recognize that various factors, such as salt conditions as well as base composition of the primer and the location of the mismatches, will affect the specificity of the primer, and that routine experimental confirmation of the primer specificity will be needed in most cases. Hybridization conditions can be chosen under which the primer can form stable duplexes only with a target sequence. Thus, the use of target-specific primers under suitably stringent amplification conditions enables the specific amplification of those target sequences which contain the target primer binding sites. The use of sequence-specific amplification conditions enables the specific amplification of those target sequences which contain the exactly complementary primer binding sites.

As used herein, “complementary” refers to a nucleic acid molecule that can form hydrogen bond(s) with another nucleic acid molecule by either traditional Watson-Crick base pairing or other non-traditional types of pairing (e.g., Hoogsteen or reversed Hoogsteen hydrogen bonding) between complementary nucleosides or nucleotides.

It is understood in the art that a nucleic acid molecule need not be 100% complementary to a target nucleic acid sequence to be specifically hybridizable. That is, two or more nucleic acid molecules may be less than fully complementary and is indicated by a percentage of contiguous residues in a nucleic acid molecule that can form hydrogen bonds with a second nucleic acid molecule. For example, if a first nucleic acid molecule has 10 nucleotides and a second nucleic acid molecule has 10 nucleotides, then base pairing of 5, 6, 7, 8, 9, or 10 nucleotides between the first and second nucleic acid molecules represents 50%, 60%, 70%, 80%, 90%, and 100% complementarity, respectively. “Perfectly” or “fully” complementary nucleic acid molecules means those in which all the contiguous residues of a first nucleic acid molecule will hydrogen bond with the same number of contiguous residues in a second nucleic acid molecule, wherein the nucleic acid molecules either both have the same number of nucleotides (i.e., have the same length) or the two molecules have different lengths.

The term “non-specific amplification,” as used herein, refers to the amplification of nucleic acid sequences other than the target sequence which results from primers hybridizing to sequences other than the target sequence and then serving as a substrate for primer extension. The hybridization of a primer to a non-target sequence is referred to as “non-specific hybridization” and is apt to occur especially during the lower temperature, reduced stringency, pre-amplification conditions.

The term “reaction mixture,” as used herein, refers to a solution containing reagents necessary to carry out a given reaction. An “amplification reaction mixture”, which refers to a solution containing reagents necessary to carry out an amplification reaction, typically contains oligonucleotide primers and a DNA polymerase or ligase in a suitable buffer. A “PCR reaction mixture” typically contains oligonucleotide primers, a DNA polymerase (most typically a thermostable DNA polymerase), dNTPs, and a divalent metal cation in a suitable buffer. A reaction mixture is referred to as complete if it contains all reagents necessary to enable the reaction, and incomplete if it contains only a subset of the necessary reagents. It will be understood by one of skill in the art that reaction components are routinely stored as separate solutions, each containing a subset of the total components, for reasons of convenience, storage stability, or to allow for application-dependent adjustment of the component concentrations, and that reaction components are combined prior to the reaction to create a complete reaction mixture. Furthermore, it will be understood by one of skill in the art that reaction components are packaged separately for commercialization and that useful commercial kits may contain any subset of the reaction components which includes the blocked primers of the disclosure.

For the purposes of this disclosure, the term “activated,” as used herein, refers to a primer or other oligonucleotide that is capable of participating in a reaction with DNA polymerase or DNA ligase. A primer or other oligonucleotide becomes activated when it hybridizes to a substantially complementary nucleic acid sequence and is chemically modified so that it can interact with a DNA polymerase or a DNA ligase. For example, when the oligonucleotide is a primer, and the primer is hybridized to a template, a 3′-blocking group can be removed from the primer by, for example, a cleaving enzyme such that DNA polymerase can bind to the 3′ end of the primer and promote primer extension.

The term “fluorescent generation probe” refers either to a) an oligonucleotide having an attached fluorophore and quencher, and optionally a minor groove binder or to b) a DNA binding reagent such as Sybr® green dye.

The terms “fluorescent label” or “fluorophore” refers to compounds with a fluorescent emission maximum between about 350 and 900 nm A wide variety of fluorophores can be used, including but not limited to: The ATTO Dyes, such as Atto647N; the black hole quencher dyes, such as BHQ2; 5-FAM (also called 5-carboxyfluorescein; also called Spiro(isobenzofuran-1(3H), 9′-(9H)xanthene)-5-carboxylic acid,3′,6′-dihydroxy-3-oxo-6-carboxyfluorescein); 5-Hexachloro-Fluorescein ([4,7,2′,4′,5′,7′-hexachloro-(3′,6′-dipivaloyl-fluoresceinyl)-6-carboxylic acid]); 6-Hexachloro-Fluorescein ([4,7,2′,4′,5′,7′-hexachloro-(3′,6′-dipivaloylfluoresceinyl)-5-carboxylic acid]); 5-Tetrachloro-Fluorescein ([4,7,2′,7′-tetra-chloro-(3′,6′-dipivaloylfluoresceinyl)-5-carboxylic acid]); 6-Tetrachloro-Fluorescein ([4,7,2′,7′-tetrachloro-(3′,6′-dipivaloylfluoresceinyl)-6-carboxylic acid]); 5-TAMRA (5-carboxytetramethylrhodamine; Xanthylium, 9-(2,4-dicarboxyphenyl)-3,6-bis(dimethyl-amino); 6-TAMRA (6-carboxytetramethylrhodamine; Xanthylium, 9-(2,5-dicarboxyphenyl)-3,6-bis(dimethylamino); EDANS (5-((2-aminoethyl)amino)naphthalene-1-sulfonic acid); 1,5-IAEDANS (5-((((2-iodoacetyl)amino)ethyl)amino)naphthalene-1-sulfonic acid); DABCYL (4-((4-(dimethylamino)phenyl)azo)benzoic acid) Cy5 (Indodicarbocyanine-5) Cy3 (Indo-dicarbocyanine-3); and BODIPY FL (2,6-dibromo-4,4-difluoro-5,7-dimethyl-4-bora-3a,4a-diaza-s-indacene-3-proprionic acid), Quasar-670 (Biosearch Technologies), CalOrange (Biosearch Technologies), Rox, as well as suitable derivatives thereof.

The term “ligation” as used herein refers to the covalent joining of two polynucleotide ends. In various embodiments, ligation involves the covalent joining of a 3′ end of a first polynucleotide (the acceptor) to a 5′ end of a second polynucleotide (the donor). Ligation results in a phosphodiester bond being formed between the polynucleotide ends. In various embodiments, ligation may be mediated by any enzyme, chemical, or process that results in a covalent joining of the polynucleotide ends. In certain embodiments, ligation is mediated by a ligase enzyme.

As used herein, “ligase” refers to an enzyme that is capable of covalently linking the 3′ hydroxyl group of a nucleotide to the 5′ phosphate group of a second nucleotide. Examples of ligases include E. coli DNA ligase, T4 DNA ligase, etc.

The ligation reaction can be employed in DNA amplification methods such as the “ligase chain reaction” (LCR), also referred to as the “ligase amplification reaction” (LAR), see Barany (1991) Proc. Natl. Acad. Sci. U.S.A. 88:189; and Wu and Wallace (1989) Genomics 4:560, incorporated herein by reference. In LCR, four oligonucleotides, two adjacent oligonucleotides which uniquely hybridize to one strand of the target DNA, and a complementary set of adjacent oligonucleotides, that hybridize to the opposite strand are mixed and DNA ligase is added to the mixture. Provided that there is complete complementarity at the junction, ligase will covalently link each set of hybridized molecules. Importantly, in LCR, two probes are ligated together only when they base-pair with sequences in the target sample, without gaps or mismatches. Repeated cycles of denaturation, hybridization and ligation amplify a short segment of DNA. LCR has also been used in combination with PCR to achieve enhanced detection of single-base changes, see Segev PCT Pub. No. WO/9001069.

As used herein, the term “conserved region” or “conserved sequence” refers to a nucleic acid sequence in a region of a gene that is the same or highly similar across different species. For example, a sequence or region of a gene that is conserved may have the same nucleic acid sequence in several types of fungal species, or, in some cases, may have the same or highly similar sequence across different taxonomic phyla (e.g., a human DNA sequence and a fungal DNA sequence in a highly conserved region of a gene may be the same or highly similar). Conversely, a “highly variable” or “hypervariable” region or sequence of gene is not conserved across species or phyla, and will have many nucleotides differences in the hypervariable region in the gene from each species.

System Process Control (SPC) Primers, Primer Sets, Compositions, and Methods

The present disclosure is based on the unexpected discovery that a control bacterium can be detected when introduced into a patient sample by using PCR primers that specifically amplify a control bacterium's DNA. Methods using the primers and primer sets provided herein uniquely detect the control bacterium's DNA and serve as a positive control for cell lysis, DNA extraction, and PCR amplification of one or more bacterium within the patient sample. Thus, the present methods are useful in a clinical setting for the rapid confirmation of the reliability of a PCR-based detection system for a bacterium in a patient sample. The primers, primer sets, and methods provided herein have both excellent analytical sensitivity and species level resolution which is specific to a bacterial species that is not present in the human.

An “individual” or “subject”, “mammal”, “patient” or “animal”, as used herein, refers to vertebrates that support a bacterial infection, including, but not limited to, birds (such as water fowl and chickens) and members of the mammalian species, such as canine, feline, lupine, mustela, rodent (racine, and murine, etc.), equine, bovine, ovine, caprine, porcine species, and primates, the latter including humans.

The term “sample” as used in the present disclosure can be any tissue, fluid, or other source of DNA from a patient or mammal A sample of this disclosure can include but is not limited to a gynecological sample (e.g., vaginal, labial, vulvar, cervical, urine, vaginal fluid, vaginal washings, vaginal secretions, vaginal tissue, anal, rectal, endometrial, fetal, placental, chorioanmiotjc, oral, salivary, skin swab or scraping, etc.), vaginal sample, labial sample, endometrial sample, cervical sample, rectal/anal sample, oral sample (e.g., saliva, tongue swab or scraping, iuner cheek swab or scraping, tooth swab or scraping), fallopian tube sample, ovary sample, peritoneal fluid or biopsy sample, anmiotic fluid sample, fetal tissue sample, placenta/chorioanmiotic tissue sample, urine sample, blood sample, plasma sample, serum sample, skin swab or sample, etc.

In certain embodiments, the SPC methods include: (a) introducing a control bacterium into a patient sample, (b) carrying out a PCR reaction on the patient sample to generate a PCR amplicon that comprises a region of the control bacterium's genome, wherein the PCR reaction uses at least one primer pair including a forward primer and a reverse primer wherein each of the forward and reverse primers is complementary to a region of the control bacterium's genome, and (c) detecting the PCR amplicon. The patient sample can, for example, be a blood sample, a sputum sample, a lung lavage fluid sample, a vaginal swab, or a tissue biopsy sample. Any fluid and/or tissue that contains, is suspected or containing, or has the potential of harboring a bacterial infection can constitute a patient sample in the present disclosure.

SPC compositions and methods disclosed herein can employ at least one forward and reverse primer pair and at least one control organism, which is not otherwise present in a patient sample. The SPC compositions and methods can also include a probe for detecting an amplified region of the genome of the at least one control organism. Exemplified herein are compositions and methods that employ at least one primer pair, and optionally at least one probe, for specifically amplifying and detecting DNA from a Megasphaera cerevisiae control bacterium, which is introduced into a patient sample prior to cell lysis and PCR amplification.

The PCR reaction carried out on the patient sample can be performed according to any of the methods known in the art. The purpose of the PCR reaction is to amplify a region of a control bacterium's DNA sequence, thereby generating a PCR amplicon. The primers and probes contemplated by the present disclosure target this region without cross-reacting with or being inhibited by the presence of a non-control bacterial DNA and/or a human DNA. Moreover, each primer of a primer pair in the PCR reaction specifically binds only to a control bacterium's DNA in the presence of a non-control bacterial DNA and/or patient DNA.

PCR reactions, including qPCT reactions, can be used to detect control bacterial DNA in a sample. It will be understood that alternative methods of DNA amplification, other than PCR amplification, such as the ligase chain reaction of Nucleic Acid Sequence Based Amplification (NASBA), can also be used to detect the presence of a control bacterium's DNA in a patient sample. Any method suitable for amplifying a region of a control bacterium's rRNA gene region is contemplated by the present disclosure.

By the presently disclosed SPC methods, the control bacterium can be a Megasphaera cerevisiae species. Sequences that are described in further detail herein and that exemplify certain aspects of the present disclosure are presented in Table 1.

TABLE 1
Sequences of Megasphaera cerevisiae Primers, Probes,
and ITS Region of the rRNA Gene

| Sequence | | |
|---|---|---|
| Identifier | Description | Nucleotide Sequence |
| SEQ ID NO: | Forward | 5'-CGAGTCACTTATGCCGGATAT-3' |
| 1 | Primer | |
| SEQ ID NO: | Reverse | 5'-CCTTACTGTATCTCTACTTCGC-3' |
| 2 | Primer | |
| SEQ ID NO: | Reverse | 5'-CCTAAGTGATTGGGTTGAGTC-3' |
| 3 | Primer | |
| SEQ ID NO: | Reverse | 5'-TTGGTTGTTCCAGAATGCCGA-3' |
| 4 | Primer | |
| SEQ ID NO: | Reverse | 5'-TGATTCATTCCAGATGAGAGAAG-3' |
| 5 | Primer | |
| SEQ ID NO: | Reverse | 5'-TGTCTTCACCTTGTATATATTAGAG-3' |
| 6 | Primer | |
| SEQ ID NO: | Probe | 5'-ATAGTATATGTTGAAAGACATGTAGTATGAGCGCAG-3' |
| 7 | | |
| SEQ ID NO: | ITS Region | GCTTGGGCTACACACGTACTACAATGGCTCTTAATAGAGGGANGCAAAGG |
| 8 | | AGCGATCCGGAGCAAACCCCAAAAACAGAGTCCCAGTTCGGATTGCAGGC |
| | | TGCAACTCGCCTGCATGAAGCAGGAATCGCTAGTAATCGCAGGTCAGCAT |
| | | ACTGCGGTGAATACGTTCCCGGGCCTTGTACACACCGCCCGTCACACCAC |
| | | GAAAGTCATTCACACCCGAAGCCGGTGAGGTAACCGCAAGGAGCCAGCCG |
| | | TCGAAGGTGGGGGCGATGATTGGGG~TGAAGTCGTAACAAGGTNNNNNTA |
| | | TCGGAAGGTGCGGCTGGATCACCTCCTTTCTAGGGATAAGCAGGGAGCCG |
| | | CATGTGAAGGAGTGCAACGCACGACGCACACATGTGAGCGAGTCACTTAT |
| | | GCCGGATATGACTGATAGTATATGTTGAAAGACATGTAGTATGAGCGCAG |
| | | ATCCGGCGAAGTAGAGATACAGTAAGGGACTCAACCCAATCACTTAGGTC |
| | | GGCATTCTGGAACAACCAATTTTGCATTGTTTAGTTTTGAGAGGCCATGT |
| | | GCTTCTCTCATCTGGAATGAATCANATGAAGCTCGTACCTTGACAACTGC |
| | | ATAGAAAAGTATTGAAGAAAGTACCTCTTTTTTTGTAAGAAAAAAGGGAG |
| | | TACCTTAACGAACCGAAGAAACTCTAATATATACAAGGTGAAGACAGCAG |
| | | GAGCCGCANGGTGAGAGCTGATCTTAAAAAGT~AAGTCAAGTAAGTAAGG |
| | | GCATACGGCGGATGCCTTGGCCATATCAGCCGAAGAAGGACGCGATAAGC |
| | | TGCGAAA |

The region of the control bacterium's genome that is amplified by the SPC methods can be at least a portion of the control bacterium's ribosomal RNA (rRNA). Within certain aspects of these methods, the amplified region of the control bacterium's rRNA gene can include at least a portion of an internal transcribed spacer (ITS) region. The amplified region of the control bacterium's rRNA gene region can, optionally, also include at least a portion of a 16S rRNA gene and/or at least a portion of a 23S rRNA gene. Typically, the PCR amplicon amplified by the present SPC methods is between about 50 bp and about 1000 bp, or between about 60 bp and about 600 bp, or between about 70 bp and about 400 bp, or between about 80 bp and about 300 bp.

The PCR amplicon can be detected by hybridization of the amplicon to a probe, such as a radiolabeled or a fluorescently labeled probe. In compositions and methods in which the control bacterium is a Megasphaera cerevisiae species and the region of the control bacterium's genome that is amplified by the PCR reaction is at least a portion of the control bacterium's ribosomal RNA (rRNA), the probe can include the nucleotide sequence presented in SEQ ID NO: 7 (i.e. 5′-ATAGTATATGTTGAAAGACATGTAGTATGAGCGCAG-3′).

The present SPC methods can, optionally, include sequencing at least a portion of the PCR amplicon that is generated by the PCR reaction. Sequencing of the PCR amplicon can be carried out according to any methods known in the art suitable for determining the sequence of a PCR amplicon.

The forward and reverse primers used in the presently disclosed methods can both be complementary to a control bacterium's ITS rRNA gene. In other aspects, the forward primer can be complementary to a control bacterium's ITS rRNA gene and the reverse primer can be complementary to a control bacterium's 23S rRNA gene. In other aspects, the forward primer can be complementary to a control bacterium's 16S rRNA gene and the reverse primer can be complementary to a control bacterium's ITS rRNA gene. In other aspects, the forward primer can be complementary to a control bacterium's 16S rRNA gene and the reverse primer can be complementary to a control bacterium's 23S rRNA gene.

In compositions and methods in which the control bacterium is a Megasphaera cerevisiae species and the region of the control bacterium's genome that is amplified by the PCR reaction is at least a portion of the control bacterium's ribosomal RNA (rRNA), the forward primer and the reverse primer can be complementary to at least a portion of the Megasphaera cerevisiae rRNA ITS region presented herein as SEQ ID NO: 8. Within these aspects of the presently disclosed compositions and methods, the portion of the Megasphaera cerevisiae rRNA ITS region that is amplified can be between about 50 bp and about 1000 bp, or between about 60 bp and about 600 bp, or between about 70 bp and about 400 bp, or between about 80 bp and about 300 bp of the sequence presented herein as SEQ ID NO: 8.

For example, the forward primer can comprise at least a portion of the nucleotide sequence 5′-CGAGTCACTTATGCCGGATAT-3′ (SEQ ID NO: 1) and/or the reverse primer can comprise at least a portion of a nucleotide sequence selected from 5′-CCTTACTGTATCTCTACTTCGC-3′ (SEQ ID NO: 2), 5′-CCTAAGTGATTGGGTTGAGTC-3′ (SEQ ID NO: 3), 5′-TTGGTTGTTCCAGAATGCCGA-3′ (SEQ ID NO: 4), 5′-TGATTCATTCCAGATGAGAGAAG-3′ (SEQ ID NO: 5), and 5′-TGTCTTCACCTTGTATATATTAGAG-3′ (SEQ ID NO: 6). Other forward and reverse primer sequences that can be used to amplify at least a portion of a Megasphaera cerevisiae rRNA ITS region are also contemplated by the present disclosure.

Within certain embodiments disclosed herein, the compositions and methods employ primer sets that include a forward and reverse primer pair wherein the primer sets can be selected from: (SEQ ID NO: 1 and SEQ ID NO: 2), (SEQ ID NO: 1 and SEQ ID NO: 3), (SEQ ID NO: 1 and SEQ ID NO: 4), (SEQ ID NO: 1 and SEQ ID NO: 5), and (SEQ ID NO: 1 and SEQ ID NO: 6). Within related aspects of these embodiments, wherein each primer of a primer pair can contain at least a portion of each of the recited sequences. For example, each primer of a primer pair can contain at least 12, 13, 14, 15, 16, 17, 18, 19, 20, or 21 contiguous nucleotides of each of the recited sequences. Alternatively or additionally, each primer of a primer pair can be at least about 80% identical with, at least about 85% identical with, at least about 90% identical with, at least about 95% identical with, and/or at least about 98% identical with each of the recited sequences.

Primers disclosed herein were designed to be used in PCR-based methods for detecting a control bacterium's DNA in a patient sample. Thus, these primers specifically bind to a control bacterium's DNA but not to DNA, including non-control bacterial DNA, which is present in a patient sample. Thus, each primer of the primer pair specifically binds only to a control bacterium's DNA in the presence of a non-control bacterium's DNA and/or a patient's DNA, which is present in a patient sample. As demonstrated herein, primers of the present disclosure permit the amplification of a control bacterium's DNA in a patient sample wherein the non-control bacterium's DNA and/or the patient's DNA is present in greater than 100,000-fold, 500,000-fold, 2,500,000-fold, or 10,000,000-fold mass excess over the amount of control bacterium's DNA.

It is to be understood in the present disclosure that any of the primer sequences disclosed herein may be modified without departing from the intended scope of the disclosure. Specifically, nucleotide substitutions, deletions and/or additions may be introduced into any of the primer sequences disclosed herein without altering the ability of the primers to identify Megasphaera cerevisiae introduced into a patient sample. Moreover, it is to be understood that the lengths of the primers may be shorter or longer than the sequences disclosed herein.

In certain embodiments, the methods described herein may be used to detect a bacterial species not specifically disclosed herein and from newly identified bacterial species. In other words, the methods provided herein can be modified for detecting other bacterial species, and are not limited to the specific examples of Megashpera cerevisiae species disclosed herein.

Also disclosed herein are methods for identifying a primer set capable of detecting a bacterial species introduced into a patient sample, the method including the steps of: (a) obtaining the DNA sequence of at least the ITS region of a bacterial rRNA operon, (b) designing a forward primer capable of hybridizing with the DNA sequence, (c) designing a reverse primer capable of hybridizing with the DNA sequence at a region in the DNA that is 3′ to the region to which the forward primer is capable of hybridizing, (d) testing whether the forward and reverse primers are capable of generating a PCR amplicon that is useful for identifying bacterial DNA using a PCR reaction containing a bacterial species.

In certain embodiments, the method also includes the steps of testing the forward and reverse primers in a PCR reaction containing a bacterial species and a patient sample. In yet other embodiments, the method includes running the PCR amplicon on an agarose gel and determining the product size. In still other embodiments, the method includes sequencing the PCR amplicon.

In yet other embodiments, the analytical sensitivity and cross-reactivity of a specific primer set may be determined by testing the specific primer set on a panel of individual samples, each sample containing a bacterial species introduced into a patient sample. An amplicon is generated by each PCR reaction containing the bacterial species and the patient sample. Each amplicon is then sequenced and the sequences of each amplicon are compared.

All U.S. patents, U.S. patent application publications, U.S. patent applications, foreign patents, foreign patent applications, non-patent publications, figures, tables, and websites referred to in this specification are expressly incorporated herein by reference, in their entirety.

The present disclosure is not to be limited in scope by the specific embodiments described herein. Indeed, various modifications of the methods and compositions disclosed herein will become apparent to those skilled in the art from the foregoing description and the accompanying figures. Such modifications are intended to fall within the scope of the appended claims. It is further to be understood that all values are approximate, and are provided for description.

### EXAMPLES

### Example 1

### Amplification of M. cerevisiae Genomic DNA in Quantitative PCR Reactions Using System Process Control Primer Pairs

Five different M. cerevisiae amplicons were generated in quantitative PCR (qPCR) reactions using the same forward primer (SEQ ID NO: 1) with five different reverse primers (SEQ ID NOs. 2-6). Standard curves were generated using 1000 pg, 100 pg, 10 pg, 1 pg, 0.5 pg, and 0.1 pg of genomic DNA extracted from M. cerevisiae and the TaqMan® Fast Advanced Master Mis for the Roche LightCycler® 480 Real-Time PCR System (Roche LC480 mastermix; Applied Biosystems, Carlsbad, Calif.), with supplemental magnesium in concentrations as presented in Table 2.

TABLE 2

| | Concentration Per |
|---|---|
| Reagent | Reaction |
| LC480 Mastermix | 1 X |
| Forward Primer | 0.8 pmol/μl |
| Reverse Primer | 0.8 pmol/μl |
| dsDNA Dye (EvaGreen) | 1.2 X |
| MgCl₂ | 1.8 mM |

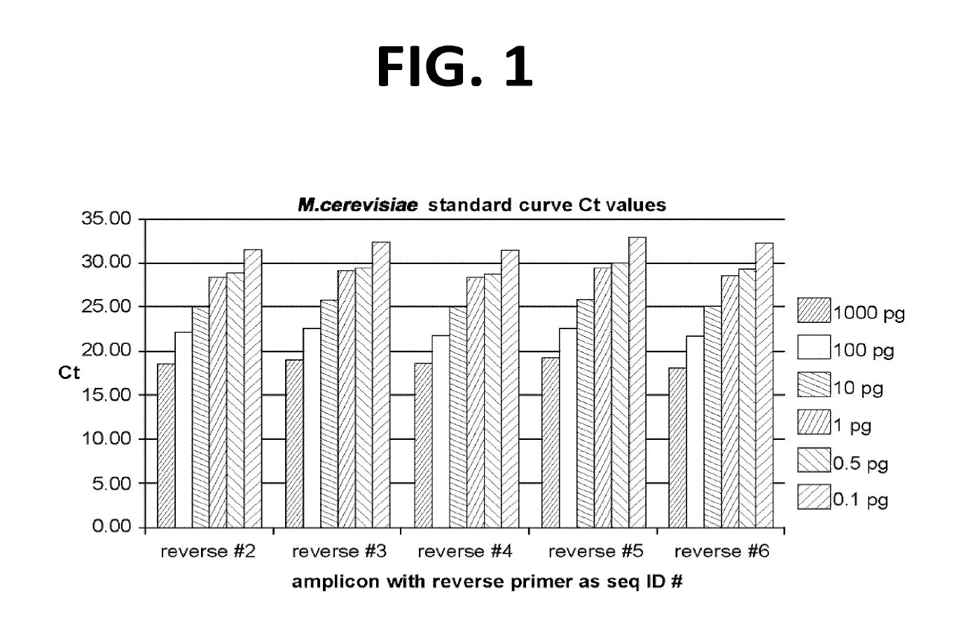

As demonstrated by the bar graph in FIG. 1, which presents threshold cycle (Ct) values as a function of M cerevisiae genomic DNA concentration, all primer combinations were able to amplify down to 0.1 pg of M. cerevisiae DNA per qPCR reaction.

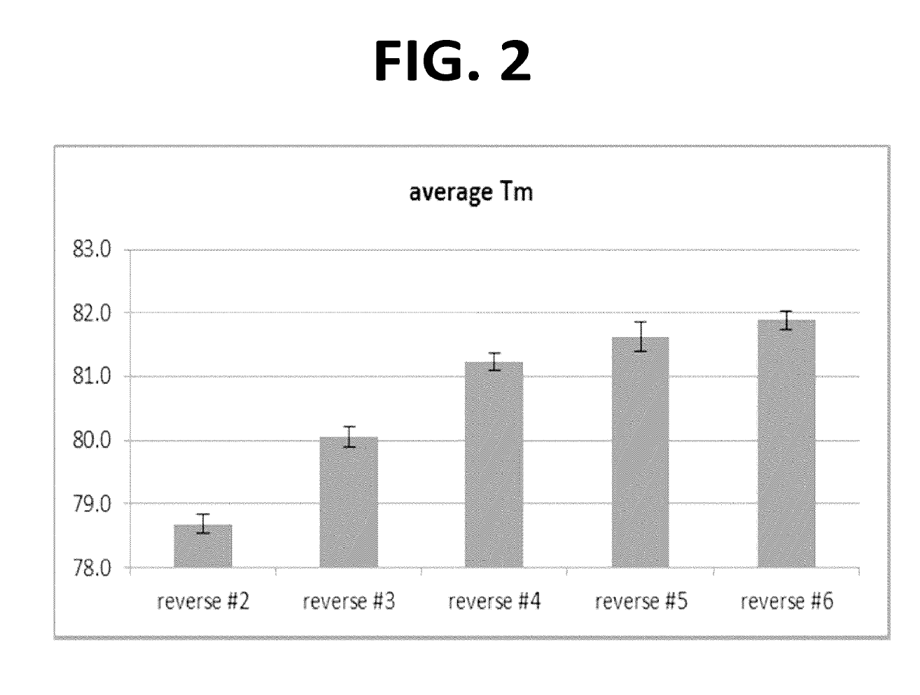

As demonstrated by the bar graph in FIG. 2, which presents average melting temperature (Tm) as a function of increasing amplicon size for quantitative PCR (qPCR) reactions using five primer combinations in which the same forward primer (SEQ ID NO: 1) was used in combination with one of five reverse primers (SEQ ID NOs: 2-6) to generate M. cerevisiae DNA amplicons, melt curve analysis confirmed that increasing Tms corresponded with increasing amplicon size.

### Example 2

### M. cerevisia System Process Control (SPC) Multiplex qPCR Reactions with Megasphaera and BVAB2 Plasmid DNAs

This Example demonstrates the detection of M cerevisiae genomic DNA in exemplary system process control (SPC) multiplex qPCR reactions for detecting Megasphaera types 1 and 2 and Clostridium-like bacterial vaginosis bacterium BVAB2 using the reagents and concentrations as presented in Table 3.

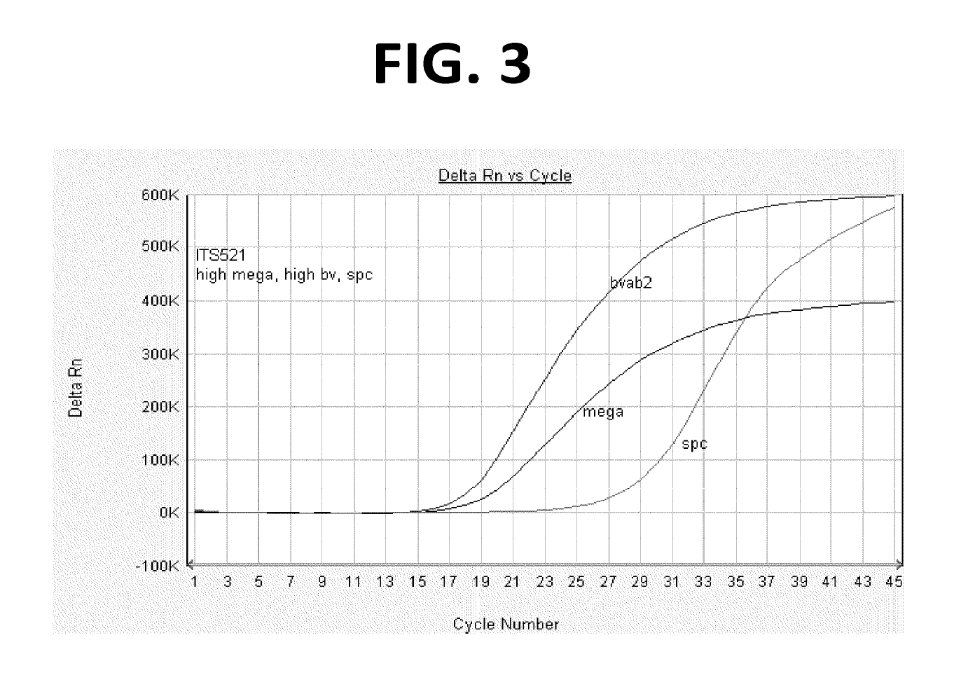

As demonstrated by the graph in FIG. 3, which shows normalized Rn (i.e., delta Rn) as a function of cycle number for amplicons derived from multiplex qPCR reactions with the reverse primer of SEQ ID NO: 4 (ITS521) and M. cerevisiae probe (SEQ ID NO: 7) in multiplex qPCR reactions targeting 0.5 pg of M. cerevisiae genomic DNA (SPC), 10⁶ 16 S plasmid copies of Megasphaera types 1 and 2 (high mega), and 10⁶ 16 S copies of the Clostridium-like bacterial vaginosis bacterium BVAB2 (high by).

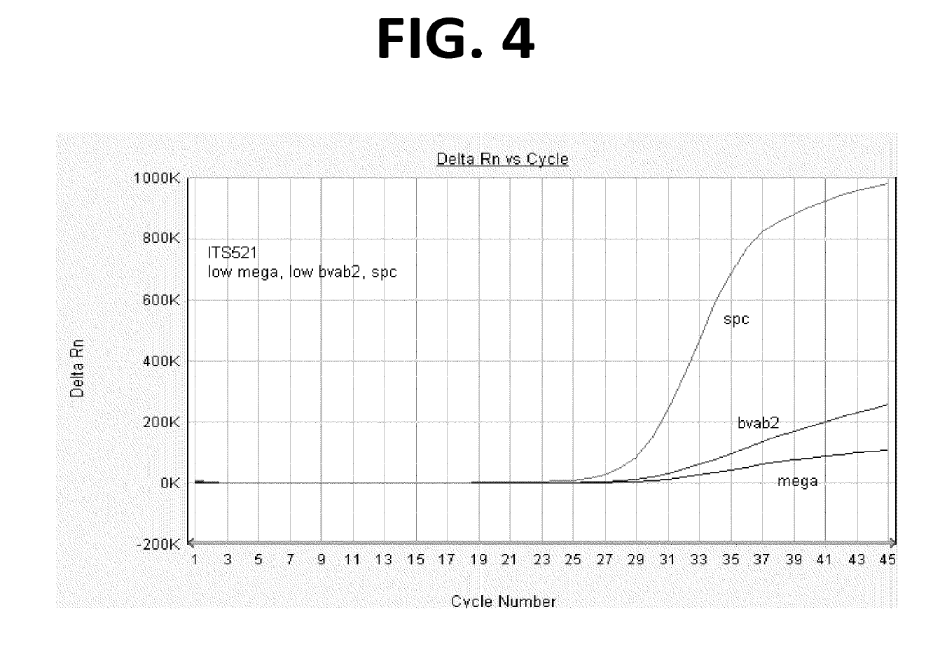

As demonstrated by the graph in FIG. 4, which shows normalized Rn (i.e., delta Rn) as a function of cycle number for amplicons derived from multiplex qPCR reactions with the reverse primer of SEQ ID NO: 4 (ITS521) and M. cerevisiae probe (SEQ ID NO: 7) in multiplex qPCR reactions targeting 0.5 pg of M. cerevisiae genomic DNA (SPC), 100 16S plasmid copies of Megasphaera types 1 and 2 (low mega), and 100 16S copies of the Clostridium-like bacterial vaginosis bacterium BVAB2 (low by).

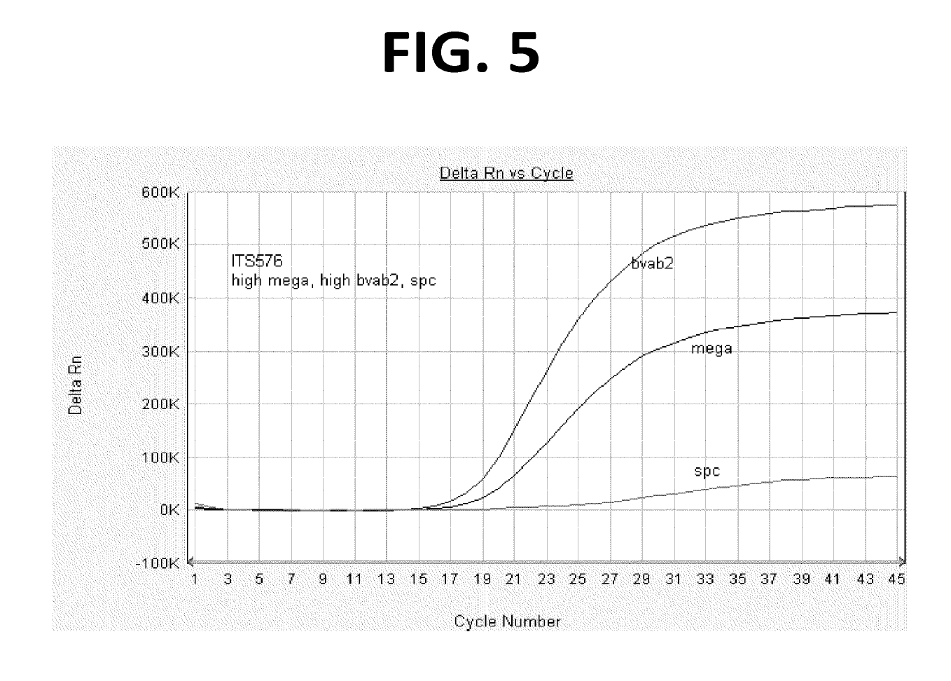

As demonstrated by the graph in FIG. 5, which shows normalized Rn (i.e., delta Rn) as a function of cycle number for amplicons derived from multiplex qPCR reactions with the reverse primer of SEQ ID NO: 5 (ITS576) and M. cerevisiae probe (SEQ ID NO: 7) in multiplex qPCR reactions targeting 0.5 pg of M. cerevisiae genomic DNA (SPC), 10⁶ 16 S plasmid copies of Megasphaera types 1 and 2 (high mega), and 10⁶ 16 S copies of the Clostridium-like bacterial vaginosis bacterium BVAB2 (high by).

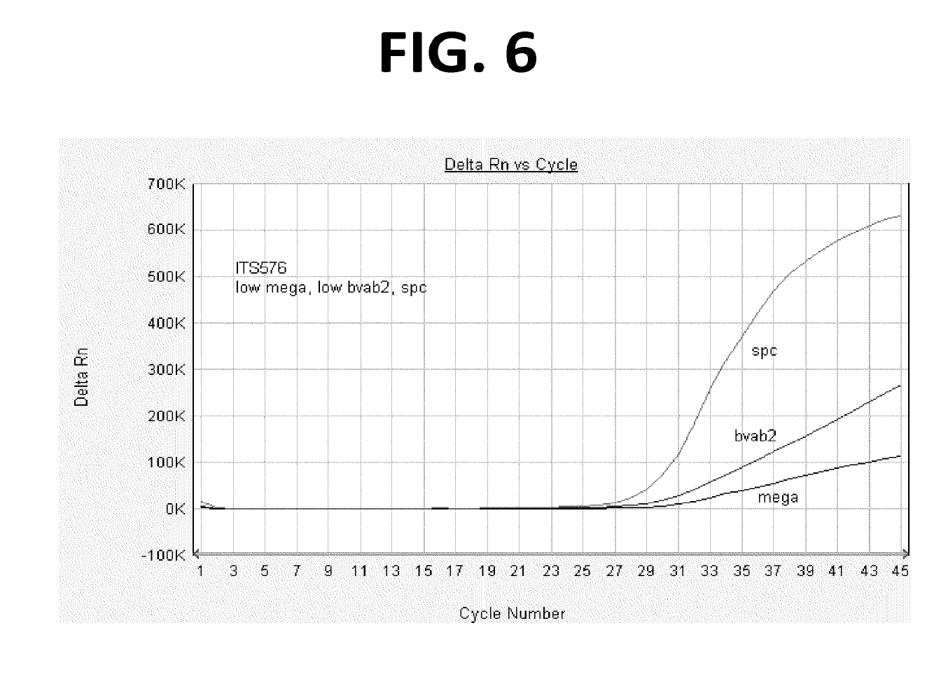

As demonstrated by the graph in FIG. 6, which shows normalized Rn (i.e., delta Rn) as a function of cycle number for amplicons derived from multiplex qPCR reactions with the reverse primer of SEQ ID NO: 5 (ITS576) and M. cerevisiae probe (SEQ ID NO: 7) in multiplex qPCR reactions targeting 0.5 pg of M. cerevisiae genomic DNA (SPC), 100 16S plasmid copies of Megasphaera types 1 and 2 (low mega), and 100 16S copies of the Clostridium-like bacterial vaginosis bacterium BVAB2 (low by).

For the multiplex qPCR experiments presented in FIGS. 3-6, delta Rn was obtained by normalizing the reporter signal (Rn) to a fluorescence signal from the ROX™ reference fluorescent dye and subtracting the baseline from Rn (i.e., ΔRn=Rn−baseline)).

TABLE 3

| | Sequence | | Concentration |
|---|---|---|---|
| Reagent | Identifier | Sequence | Per Reaction |
| LC480 Mastermix | | | 1 X |
| Forward Primer, SPC | SEQ ID NO: 1 | See, Table 1 | 0.4 pmol/μl |
| Reverse Primer, SPC (variable) | SEQ ID NOs: 2-6 | See, Table 1 | 0.4 pmol/μl |
| Probe, SPC | SEQ ID NO: 7 | See, Table 1 | 200 nM |
| Forward Primer, BVAB2 | SEQ ID NO: 9 | ttaaccttgg ggttcattac aa | 0.8 pmol/μl |
| Reverse Primer, BVAB2 | SEQ ID NO: 10 | aattcagtct cctgaatcgt caga | 0.8 pmol/μl |
| Probe, BVAB2 | SEQ ID NO: 11 | cgacgtcgct gctctctgtt gta | 200 nM |
| Forward Primer, Mega Type 1 | SEQ ID NO: 12 | gatgccaaca gtatccgtcc g | 0.8 pmol/μl |
| Reverse Primer, Mega Type 1 | SEQ ID NO: 13 | cctctccgac actcaagttc ga | 0.8 pmol/μl |
| Forward Primer, Mega Type 2 | SEQ ID NO: 14 | aaggtggtaa atagccatca tgag | 0.8 pmol/μl |
| Reverse Primer, Mega Type 2 | SEQ ID NO: 15 | ctctccgaca ctcaagtctt c | 0.8 pmol/μl |
| Probe, Mega Types 1 and 2 | SEQ ID NO: 16 | gtaccgtaag agaaagccac gg | 200 nM |
| MgCl₂ | | | 1.8 mM |

### Example 3

### M. cerevisia System Process Control (SPC) Multiplex qPCR Reactions with Vaginosis Bacteria from Vaginal Swab Samples

This Example demonstrates M cerevisiae system process control (SPC) specificity using reverse primers in multiplex qPCR reactions performed in the presence of vaginosis bacteria from vaginal swab.

System process control (SPC) specificity using M cerevisiae ITS521R and ITS576R reverse primers was tested in multiplex qPCR reactions by running genomic DNA extracted from vaginal swabs obtained from commercial sex workers in Mombasa, Kenya spiked with 0.5 pg M. cerevisiae DNA samples using the reagents and concentrations as presented in Table 3. DNA samples were selected according to previously-run singleplex BVAB2 and Mega type 1 and type 2 qPCR data as presented in Table 4.

TABLE 4

| | | BVAB2 Load |
|---|---|---|
| | Mega Type 1 and Type 2 | Based on |
| | Load Based on Singleplex | Singleplex |
| | qPCR Data | qPCR Data |
| Mombasa DNA Type 1 | High Positive | High Positive |
| Mombasa DNA Type 2 | Low or Negative | Low or Negative |
| Mombasa DNA Type 3 | Positive | Low or Negative |
| Mombasa DNA Type 4 | Low or Negative | Positive |

System process control (SPC) threshold cycle (Ct) values across all Mega Type 1, Mega Type 2, and BVAB2 loads remained within 0.14 Cts for M cerevisiae ITS521R reverse primer (SEQ ID NO: 4) and 0.39 Cts for M. cerevisiae ITS576R reverse primer (SEQ ID NO: 5). These data demonstrate that there was a minimal impact of Megasphaera Type 1, Megasphaera Type 2, and BVAB2 DNA on amplification of an exemplary SPC.

1. A system process control (SPC) method, comprising:
(a) introducing a control bacterium into a patient sample;
(b) carrying out a PCR reaction on the patient sample to generate a PCR amplicon that comprises a region of the control bacterium's genome, wherein the PCR reaction uses at least one primer pair including a forward primer and a reverse primer wherein each of the forward and reverse primers is complementary to a region of the control bacterium's genome; and
(c) detecting the PCR amplicon.

2. The SPC method of claim 1 wherein said PCR amplicon comprises at least a portion of the control bacterium's ribosomal RNA (rRNA).

3. The SPC method of claim 1 wherein said PCR amplicon comprises at least a portion of an internal transcribed spacer (ITS) region of said rRNA gene.

4. The SPC method of claim 1 wherein said PCR amplicon comprises at least a portion of a 16S region of said rRNA gene.

5. The SPC method of claim 1 wherein said PCR amplicon comprises at least a portion of a 23S region of said rRNA gene.

6. The SPC method of claim 1 wherein said PCR amplicon is between about 50 bp and about 1000 bp, or between about 60 bp and about 600 bp, or between about 70 bp and about 400 bp, or between about 80 bp and about 300 bp.

7. The SPC method of claim 1 wherein said PCR amplicon is detected by hybridization of a probe.

8. The SPC method of claim 7 wherein said probe is radiolabeled or fluorescently labeled.

9. The SPC method of claim 1 wherein at least a portion of said PCR amplicon is sequenced.

10. The SPC method of any one of claims 1-9 wherein said control bacterium is Megasphaera cerevisiae.

11. The SPC method of claim 10 wherein said forward primer and said reverse primer are complementary to at least a portion of said Megasphaera cerevisiae's rRNA gene.

12. The SPC method of claim 11 wherein said forward primer and said reverse primer are complementary to at least a portion of the ITS region of said Megasphaera cerevisiae's rRNA gene wherein said ITS region comprises the sequence presented in SEQ ID NO: 8.

13. The SPC method of claim 11 wherein said forward primer is complementary to at least a portion of the ITS region of said rRNA and said reverse primer is complementary to at least a portion of the 23S region of said rRNA gene.

14. The SPC method of claim 11 wherein said forward primer is complementary to at least a portion of the 16S region of said rRNA gene and said reverse primer is complementary to at least a portion of the ITS region of said rRNA gene.

15. The SPC method of claim 11 wherein said forward primer is complementary to at least a portion of the 16S region of said rRNA gene and said reverse primer is complementary to at least a portion of the 23S region of said rRNA gene.

16. The SPC method of claim 10 wherein said PCR amplicon is detected by hybridization of a probe comprising the nucleotide sequence presented in SEQ ID NO: 7.

17. The SPC method of claim 10 wherein said PCR amplicon comprises between about 50 bp and about 1000 bp, or between about 60 bp and about 600 bp, or between about 70 bp and about 400 bp, or between about 80 bp and about 300 bp of the sequence presented herein as SEQ ID NO: 8.

18. The SPC method of claim 10 wherein said forward primer comprises at least a portion of the nucleotide sequence of SEQ ID NO: 1.

19. The SPC method of claim 10 wherein said reverse primer comprises at least a portion of a nucleotide sequence selected from SEQ ID NO: 2, SEQ ID NO: 3, SEQ ID NO: 4, SEQ ID NO: 5, and SEQ ID NO: 6.

20. The SPC method of claim 10 wherein said forward primer and said reverse primer comprise a primer set selected from (SEQ ID NO: 1 and SEQ ID NO: 2), (SEQ ID NO: 1 and SEQ ID NO: 3), (SEQ ID NO: 1 and SEQ ID NO: 4), (SEQ ID NO: 1 and SEQ ID NO: 5), and (SEQ ID NO: 1 and SEQ ID NO: 6).

21-56. (canceled)


SEQUENCE LISTING (16 sequences)

SEQ ID NO: 1 - DNA, 21 bp, Artificial Sequence
  Forward Primer, SPC
cgagtcacttatgccggatat

SEQ ID NO: 2 - DNA, 22 bp, Artificial Sequence
  Reverse Primer, SPC (variable)
ccttactgtatctctacttcgc

SEQ ID NO: 3 - DNA, 21 bp, Artificial Sequence
  Reverse Primer, SPC (variable)
cctaagtgattgggttgagtc

SEQ ID NO: 4 - DNA, 21 bp, Artificial Sequence
  Reverse Primer, SPC (variable)
ttggttgttccagaatgccga

SEQ ID NO: 5 - DNA, 23 bp, Artificial Sequence
  Reverse Primer, SPC (variable)
tgattcattccagatgagagaag

SEQ ID NO: 6 - DNA, 25 bp, Artificial Sequence
  Reverse Primer, SPC (variable)
tgtcttcaccttgtatatattagag

SEQ ID NO: 7 - DNA, 36 bp, Artificial Sequence
  Probe, SPC
atagtatatgttgaaagacatgtagtatgagcgcag

SEQ ID NO: 8 - DNA, 805 bp, Megasphaera cerevisiae
  misc_feature (43)..(43): n is a, c, g, or t
gcttgggctacacacgtactacaatggctcttaatagagggangcaaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggtaaccgcaaggagccagccgtcgaaggtgggggcgatgattggggtgaagtcgtaacaaggtnnnnntatcggaaggtgcggctggatcacctcctttctagggataagcagggagccgcatgtgaaggagtgcaacgcacgacgcacacatgtgagcgagtcacttatgccggatatgactgatagtatatgttgaaagacatgtagtatgagcgcagatccggcgaagtagagatacagtaagggactcaacccaatcacttaggtcggcattctggaacaaccaattttgcattgtttagttttgagaggccatgtgcttctctcatctggaatgaatcanatgaagctcgtaccttgacaactgcatagaaaagtattgaagaaagtacctctttttttgtaagaaaaaagggagtaccttaacgaaccgaagaaactctaatatatacaaggtgaagacagcaggagccgcanggtgagagctgatcttaaaaagtaagtcaagtaagtaagggcatacggcggatgccttggccatatcagccgaagaaggacgcgataagctgcgaaa

SEQ ID NO: 9 - DNA, 22 bp, Artificial Sequence
  Forward Primer, BVAB2
ttaaccttggggttcattacaa

SEQ ID NO: 10 - DNA, 24 bp, Artificial Sequence
  Reverse Primer, BVAB2
aattcagtctcctgaatcgtcaga

SEQ ID NO: 11 - DNA, 23 bp, Artificial Sequence
  Probe, BVAB2
cgacgtcgctgctctctgttgta

SEQ ID NO: 12 - DNA, 21 bp, Artificial Sequence
  Forward Primer, Mega Type 1
gatgccaacagtatccgtccg

SEQ ID NO: 13 - DNA, 22 bp, Artificial Sequence
  Reverse Primer, Mega Type 1
cctctccgacactcaagttcga

SEQ ID NO: 14 - DNA, 24 bp, Artificial Sequence
  Forward Primer, Mega Type 2
aaggtggtaaatagccatcatgag

SEQ ID NO: 15 - DNA, 21 bp, Artificial Sequence
  Reverse Primer, Mega Type 2
ctctccgacactcaagtcttc

SEQ ID NO: 16 - DNA, 22 bp, Artificial Sequence
  Probe, Mega Types 1 and 2
gtaccgtaagagaaagccacgg